(19) United States
(12) Patent Application Publication
(10) Pub. No.: US 2023/0078228 A1
(43) Pub. Date: Mar. 16, 2023

(54) Blood Oxygen Saturation Measurement Method And Apparatus

(71) Applicant: Anhui Huami Health Technology Co., Ltd., Hefei (CN)

(72) Inventors: Guokang Zhu, Hefei (CN); Kongqiao Wang, Hefei (CN)

(21) Appl. No.: 17/984,756

(22) Filed: Nov. 10, 2022

Related U.S. Application Data
(63) Continuation of application No. PCT/CN2021/095859, filed on May 25, 2021.

(30) Foreign Application Priority Data
May 29, 2020 (CN) 202010476854.2

(51) Int. Cl. A61B 5/00; A61B 5/1455

(52) U.S. Cl. CPC A61B 5/6843; A61B 5/14551; A61B 5/7221; A61B 5/7225

(57) ABSTRACT

A blood oxygen saturation measurement method includes: acquiring a pressure measurement signal between a blood oxygen saturation measurement apparatus and an object; in response to determining that the pressure measurement value is within a preset range, and acquiring a blood oxygen saturation measurement signal of the object; and according to the blood oxygen saturation measurement signal of the object, obtaining a blood oxygen saturation measurement value of the object.

### CROSS-REFERENCE TO RELATED APPLICATION(S)

This application is a continuation of International Application No. PCT/CN2021/095859, filed on May 25, 2021, which claims priority to Chinese Patent Application No. 202010476854.2, filed on May 29, 2020, the entire contents of both of which are incorporated herein by reference.

### TECHNICAL FIELD

The disclosure relates to the field of medical instruments, in particular to a method and an apparatus for measuring a blood oxygen saturation, a storage medium and an electronic device.

### BACKGROUND

With the development of smart wearable device technology, it is possible to use wearable devices to perform non-invasive physiological parameter measurements. For example, non-invasive blood oxygen saturation measurement can be performed to diagnose the sleep-disordered breathing syndrome, which is widely admired by users for convenience and is of great significance in clinical practice. The blood oxygen saturation is another important vital sign index other than pulse rate, body temperature, blood pressure and respiration. The clinical significance of the non-invasive blood oxygen saturation measurement has been widely recognized.

The non-invasive blood oxygen saturation measurement is mainly based on the Lambert-Beer law, to deduce the blood oxygen saturation by measuring absorption amounts of blood for lights of two different wavelengths according to Photo PlethysmoGraphy (PPG) technology based on the principle of different spectral absorption rates of oxyhemoglobin and deoxyhemoglobin.

### SUMMARY

According to a first aspect of the disclosure, a method for measuring a blood oxygen saturation is provided. The method includes: obtaining a pressure measurement signal between a blood oxygen saturation measurement apparatus and an object, so as to obtain a pressure measurement value; in response to determining that the pressure measurement value is within a preset range, obtaining a blood oxygen saturation measurement signal of the object; and obtaining a blood oxygen saturation measurement value of the object based on the blood oxygen saturation measurement signal of the object.

According to an implementation of the disclosure, the method further includes: in response to determining that the pressure measurement value is not within the preset range, adjusting a pressure between the blood oxygen saturation measurement apparatus and the object.

According to an implementation of the disclosure, in response to determining that the pressure measurement value is not within the preset range, adjusting the pressure between the blood oxygen saturation measurement apparatus and the object, includes: in response to determining that the pressure measurement value is less than a lower limit of the preset range, increasing the pressure between the blood oxygen saturation measurement apparatus and the object; or in response to determining that the pressure measurement value is greater than an upper limit of the preset range, reducing the pressure between the blood oxygen saturation measurement apparatus and the object.

According to an implementation of the disclosure, the method further includes: obtaining a first error threshold and a second error threshold of the blood oxygen saturation measurement, in which the first error threshold is less than the second error threshold; obtaining a blood oxygen saturation measurement standard value; and determining the preset range based on a difference between the blood oxygen saturation measurement value and the measurement standard value, the first error threshold and the second error threshold.

According to an implementation of the disclosure, the preset range includes: a first preset range, a second preset range and a third preset range. An upper limit of the first preset range is equal to a lower limit of the second preset range, and an upper limit of the second preset range is equal to a lower limit of the third preset range.

According to an implementation of the disclosure, determining the first preset range includes: in response to adjusting a pressure value between the blood oxygen saturation measurement apparatus and the object, an absolute value of the difference between the blood oxygen saturation measurement value and the measurement standard value being the second error threshold, and the pressure value being inversely correlated with the absolute value, determining the pressure value as a lower limit of the first preset range; or in response to adjusting a pressure value between the blood oxygen saturation measurement apparatus and the object, an absolute value of the difference between the blood oxygen saturation measurement value and the measurement standard value being the first error threshold, and the pressure value being inversely correlated with the absolute value, determining the pressure value as the upper limit of the first preset range.

According to an implementation of the disclosure, determining the third preset range includes: in response to adjusting the pressure value between the blood oxygen saturation measurement apparatus and the object, the absolute value of the difference between the blood oxygen saturation measurement value and the measurement standard value being the first error threshold, and the pressure value being positively correlated with the absolute value, determining the pressure value as the lower limit of the third preset range; or in response to adjusting the pressure value between the blood oxygen saturation measurement apparatus and the object, the absolute value of the difference between the blood oxygen saturation measurement value and the measurement standard value being the second error threshold, and the pressure value being positively correlated with the absolute value, determining the pressure value as an upper limit of the third preset range.

According to an implementation of the disclosure, before obtaining the blood oxygen saturation measurement value of the object, the method further includes: performing signal processing on the blood oxygen saturation measurement signal, in which the signal processing includes at least one of frequency domain signal processing, time domain signal processing and space domain signal processing.

According to an implementation of the disclosure, performing the signal processing on the blood oxygen saturation measurement signal includes: obtaining a first blood oxygen saturation measurement signal with a frequency less than a frequency threshold by performing low-pass filtering on the blood oxygen saturation measurement signal; determining a second blood oxygen saturation measurement signal with a time domain stability parameter less than a stability threshold from the first blood oxygen saturation measurement signal; and determining a third blood oxygen saturation measurement signal with a signal peak amplitude parameter less than an amplitude threshold from the second blood oxygen saturation measurement signal.

According to an implementation of the disclosure, the method further includes: in response to the pressure measurement value being in the second preset range, performing a first type of signal processing on the blood oxygen saturation measurement value; or in response to the pressure measurement value being in the first preset range or the third preset range, performing a second type of signal processing on the blood oxygen saturation measurement value, wherein a frequency threshold of the first type of signal processing is greater than a frequency threshold of the second type of signal processing, a stability threshold of the first type of signal processing is greater than a stability threshold of the second type of signal processing, and an amplitude threshold of the first type of signal processing is greater than an amplitude threshold of the second type of signal processing.

According to a second aspect of the disclosure, an apparatus for measuring a blood oxygen saturation is provided. The apparatus includes: a pressure measurement module, configured to obtain a pressure measurement signal between a blood oxygen saturation measurement apparatus and an object, so as to obtain a pressure measurement value; a blood oxygen saturation measurement module, configured to obtain a blood oxygen saturation measurement signal of the object in response to determining that the pressure measurement value is within a preset range, and to obtain a blood oxygen saturation measurement value of the object based on the blood oxygen saturation measurement signal of the object.

According to an implementation of the disclosure, the apparatus further includes: an information prompt module, configured to, in response to determining that the pressure measurement value is not within the preset range, issue an information prompt signal, in which the information prompt signal is configured to instruct to adjust a pressure between the blood oxygen saturation measurement apparatus and the object.

According to an implementation of the disclosure, the information prompt module includes a first information prompt unit and a second information prompt unit. The first information prompt unit is configured to, in response to determining that the pressure measurement value is less than a lower limit of the preset range, issue a first information prompt signal, in which the first information prompt signal is configured to instruct to increase the pressure between the blood oxygen saturation measurement apparatus and the object. The second information prompt unit is configured to, in response to determining that the pressure measurement value is greater than an upper limit of the preset range, issue a second information prompt signal, in which the second information prompt signal is configured to instruct to reduce the pressure between the blood oxygen saturation measurement apparatus and the object.

According to an implementation of the disclosure, the apparatus further includes a preset range determining module. The preset range determining module is configured to: obtain a first error threshold and a second error threshold of the blood oxygen saturation measurement, in which the first error threshold is less than the second error threshold; obtain a blood oxygen saturation measurement standard value; and determine the preset range based on a difference between the blood oxygen saturation measurement value and the measurement standard value, the first error threshold and the second error threshold.

According to an implementation of the disclosure, the preset range includes: a first preset range, a second preset range and a third preset range. An upper limit of the first preset range is equal to a lower limit of the second preset range, an upper limit of the second preset range is equal to a lower limit of the third preset range.

The preset range determining module includes: a first determining unit, configured to, in response to adjusting a pressure value between the blood oxygen saturation measurement apparatus and the object, an absolute value of the difference between the blood oxygen saturation measurement value and the measurement standard value being the second error threshold, and the pressure value being inversely correlated with the absolute value, determine the pressure value as a lower limit of the first preset range; a second determining unit, configured to, in response to adjusting a pressure value between the blood oxygen saturation measurement apparatus and the object, an absolute value of the difference between the blood oxygen saturation measurement value and the measurement standard value being the first error threshold, and the pressure value being inversely correlated with the absolute value, determine the pressure value as the upper limit of the first preset range.

According to an implementation of the disclosure, the preset range determining module includes: a third determining unit, configured to, in response to adjusting the pressure value between the blood oxygen saturation measurement apparatus and the object, the absolute value of the difference between the blood oxygen saturation measurement value and the measurement standard value being the first error threshold, and the pressure value being positively correlated with the absolute value, determine the pressure value as the lower limit of the third preset range; a fourth determining unit, configured to, in response to adjusting the pressure value between the blood oxygen saturation measurement apparatus and the object, the absolute value of the difference between the blood oxygen saturation measurement value and the measurement standard value being the second error threshold, and the pressure value being positively correlated with the absolute value, determine the pressure value as an upper limit of the third preset range.

According to an implementation of the disclosure, the apparatus further includes: a determining module, configured to determine whether the pressure measurement value is within the preset range, and in response to the pressure measurement value being within the preset range, instruct the blood oxygen saturation measurement module to obtain the blood oxygen saturation measurement signal.

According to an implementation of the disclosure, the blood oxygen saturation measurement module is further configured to: perform signal processing on the blood oxygen saturation measurement signal, in which the signal processing includes at least one of frequency domain signal processing, time domain signal processing and space domain signal processing.

According to an implementation of the disclosure, the blood oxygen saturation measurement module further includes: a frequency domain signal processing unit, configured to obtain a first blood oxygen saturation measurement signal with a frequency less than a frequency threshold by performing low-pass filtering on the blood oxygen saturation measurement signal; a time domain signal processing unit, configured to determine a second blood oxygen saturation measurement signal with a time domain stability parameter less than a stability threshold from the first blood oxygen saturation measurement signal; a space domain signal processing unit, configured to determine a third blood oxygen saturation measurement signal with a signal peak amplitude parameter less than an amplitude threshold from the second blood oxygen saturation measurement signal; and a signal conversion unit, configured to obtain the blood oxygen saturation measurement value based on the third blood oxygen saturation measurement signal.

According to an implementation of the disclosure, the pressure measurement module is configured to, in response to the pressure measurement value being in the second preset range, instruct the blood oxygen saturation measurement module to perform a first type of signal processing on the blood oxygen saturation measurement value. The pressure measurement module is configured to, in response to the pressure measurement value being in the first preset range or the third preset range, instruct the blood oxygen saturation measurement module to perform a second type of signal processing on the blood oxygen saturation measurement value.

The blood oxygen saturation measurement module is configured such that, a frequency threshold of the first type of signal processing is greater than a frequency threshold of the second type of signal processing, a stability threshold of the first type of signal processing is greater than a stability threshold of the second type of signal processing, and an amplitude threshold of the first type of signal processing is greater than an amplitude threshold of the second type of signal processing.

According to a third aspect of the disclosure, a computer storage medium having computer programs stored thereon is provided. When the computer programs are executed by a processor, the above blood oxygen saturation measurement method is implemented.

According to a fourth aspect of the disclosure, an electronic device is provided. The electronic device includes a memory, a processor, and computer programs stored on the memory and capable of running on the processor. When the computer programs are executed by a processor, the above blood oxygen saturation measurement method is implemented.

The above technical solution of implementations of the disclosure has the following beneficial technical effects.

According to the method and apparatus for measuring a blood oxygen saturation of the implementations of the disclosure, the pressure measurement value between the blood oxygen saturation measurement apparatus and the object is obtained, and the pressure value between the blood oxygen saturation measurement apparatus and the object is adjusted when the pressure measurement value is not in the preset range, and the blood oxygen saturation information is acquired when the pressure measurement value is within the preset range, which avoids the acquisition of erroneous blood oxygen saturation information, and improves the accuracy of measurement by the blood oxygen saturation measurement apparatus.

### BRIEF DESCRIPTION OF DRAWINGS

FIG. 1a is a schematic diagram of principle of a transmission oximeter according to an implementation of the disclosure.

FIG. 1b is a schematic diagram of principle of a reflection oximeter according to an implementation of the disclosure.

FIG. 2 is a flowchart of a method for measuring a blood oxygen saturation according to an implementation of the disclosure.

FIG. 3 is a schematic diagram of modules of an apparatus for measuring a blood oxygen saturation according to an implementation of the disclosure.

FIG. 4 is a schematic diagram of an information prompt module according to an implementation of the disclosure.

FIG. 5 is a schematic diagram of a preset range determining module according to an implementation of the disclosure.

FIG. 6 is a schematic diagram of a blood oxygen saturation measurement module according to an implementation of the disclosure.

### REFERENCE NUMBERS

pressure measurement module 1;
blood oxygen saturation measurement module 2;
frequency domain signal processing unit 21;
time domain signal processing unit 22;
space domain signal processing unit 23;
signal conversion unit 24;
information prompt module 3;
first information prompt unit 31;
second information prompt unit 32;
preset range determining module 4;
first determining unit 41;
second determining unit 42;
third determining unit 43;
fourth determining unit 44;
determining module 5

### DETAILED DESCRIPTION

In order to make the objectives, technical solutions and advantages of the disclosure clearer, the disclosure will be further described in detail below with reference to the specific implementations and the accompanying drawings. It should be understood that these descriptions are exemplary only and are not intended to limit the scope of the disclosure. In addition, descriptions of well-known structures and techniques are omitted to avoid unnecessarily obscuring the concepts of the disclosure.

The purpose of implementations of the disclosure is to provide a method for measuring a blood oxygen saturation, which obtains a pressure measurement value between the blood oxygen saturation measurement apparatus and an object, determines whether the pressure measurement value is in a preset range, does not collect blood oxygen saturation information temporarily when a pressure between the blood oxygen saturation measurement apparatus and the object is too large or too small, and prompts the user to intervene in the measurement state, and collects the blood oxygen saturation information when the pressure measurement value is within the preset range, which avoids the acquisition of erroneous blood oxygen saturation information, and improves the accuracy of measurement by the blood oxygen saturation measurement apparatus.

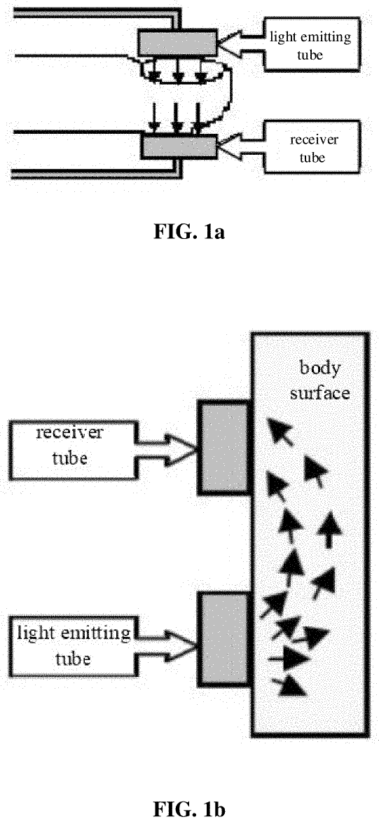

Generally, two LEDs of different wavelengths are used as light sources, and phototransistors or other photosensitive devices are used to collect signals. There are mainly two methods of transmission method and reflection method. As illustrated in FIG. 1a, the main structure difference between the transmission oximeter and the reflection oximeter is different relative positions of the light source and the sensor. The sensor and the light source of the transmission oximeter may be placed on different sides of the human tissue, and the light emitted from the light source travels through the tissue to the sensor. As illustrated in FIG. 1B, the sensor and the light source of the reflection oximeter are arranged on the same side of the tissue. After the light enters the tissue, it is reflected, scattered and then returned to be collected by the sensor.

When the transmission oximeter measures the blood oxygen saturation, according to existing solutions, it must be ensured that the light can penetrate through the human tissue and generally act on fingers, toes, earlobes and nose. However, measurements on the above spots tend to limit the daily activities of users, which may cause discomfort. The reflection oximeter has less restrictions on the measurement spots, and can be worn on the wrist, arm, leg, forehead and most parts of the body, which has less interference on daily activities and has better portability.

However, the disclosure has come to the conclusion in the research process that the contact pressure between the device and the body tissue during the reflection measurement has a great influence on the measurement result. When the sensor is loosely attached to the body tissue (for example, there is a gap between the sensor and the body tissue), or the sensor is tightly attached to the body tissue and the body tissue is squeezed, the light signal cannot reliably reflect the blood oxygen saturation signs, and the existing devices cannot cope with these conditions and cannot guarantee correct measurement of the blood oxygen saturation.

FIG. 2 is a flowchart of a method for measuring a blood oxygen saturation according to an implementation of the disclosure.

As illustrated in FIG. 2, the first aspect of the implementations of the disclosure provides a method for measuring a blood oxygen saturation. The method includes: obtaining a pressure measurement signal between a blood oxygen saturation measurement apparatus and an object, so as to obtain a pressure measurement value; in response to determining that the pressure measurement value is within a preset range, obtaining a blood oxygen saturation measurement signal of the object; and obtaining a blood oxygen saturation measurement value of the object based on the blood oxygen saturation measurement signal of the object.

Optionally, the pressure measurement value between the blood oxygen saturation measurement apparatus and the object may be obtained through a pressure measurement module of the blood oxygen saturation measurement apparatus, or may be obtained in other ways, which is not limited in the disclosure, as long as the acquisition of the pressure value between the blood oxygen saturation measurement apparatus and the object can be achieved, belongs to the protection scope of the disclosure.

According to an implementation of the disclosure, in response to determining that the pressure measurement value is not within the preset range, the pressure between the blood oxygen saturation measurement apparatus and the object is adjusted. For example, in response to determining that the pressure measurement value is less than a lower limit of the preset range, the pressure between the blood oxygen saturation measurement apparatus and the object is increased; or in response to determining that the pressure measurement value is greater than an upper limit of the preset range, the pressure between the blood oxygen saturation measurement apparatus and the object is reduced.

In some implementations, the preset range includes: a first preset range, a second preset range and a third preset range. An upper limit of the first preset range is equal to a lower limit of the second preset range, and an upper limit of the second preset range is equal to a lower limit of the third preset range. The first preset range and the third preset range are measurable ranges, and the second preset range is an ideal pressure range. In detail, the first preset range and the third preset range may be referred to as the measurable pressure range, and the second preset range may be referred to as the ideal pressure range. In other implementations, the ideal pressure range and the measurable pressure range can be divided in other manners. In addition, if the pressure measurement value is not in the preset range, it is determined to be within a measurement rejection range and the blood oxygen saturation measurement is not performed.

Optionally, the meaning of the ideal pressure range is that if the pressure measurement value obtained by the blood oxygen saturation measurement apparatus is within the ideal pressure range, an error of the blood oxygen saturation obtained by further measurement is relatively small, which is less than a preset first error threshold. That is, when the pressure value measured by the blood oxygen saturation measurement apparatus is within this ideal pressure range, the measured blood oxygen saturation signal has little noise and the measured blood oxygen saturation value determined by the blood oxygen saturation signal is relatively accurate.

It can be understood that the meaning of the above measurable pressure range is that if the pressure measurement value measured by the blood oxygen saturation measurement apparatus is in the measurable pressure range, the error of the blood oxygen saturation measured by the blood oxygen saturation measurement apparatus is within an acceptable range, for example, the error is greater than a preset first error threshold but less than a preset second error threshold. That is, if the pressure measurement value obtained by the blood oxygen saturation measurement apparatus is within the measurable pressure range, the error can be reduced by processing the measured blood oxygen saturation signal.

Optionally, the first preset range ranges from the first preset threshold to the second preset threshold. The second preset range ranges from the second preset threshold and the third preset threshold. The third preset range ranges from the third preset threshold to the fourth preset threshold.

According to an implementation of the disclosure, the method further includes: in response to determining that the pressure measurement value is not within the preset range, outputting a pressure adjustment signal, wherein the pressure adjustment signal is configured to instruct to adjust the pressure between the blood oxygen saturation measurement apparatus and the object.

In some implementations, the pressure adjustment signal includes a first pressure adjustment signal or a second pressure adjustment signal. The first pressure adjustment signal is used for instructing to increase the pressure between the blood oxygen saturation measurement apparatus and the object. The second pressure adjustment signal is used for instructing to decrease the pressure between the blood oxygen saturation measurement apparatus and the object.

Optionally, in response to determining that the pressure measurement value is less than the lower limit of the preset range, e.g., the first preset threshold, the first pressure adjustment signal is sent. The first pressure adjustment signal is configured to prompt that the blood oxygen saturation measurement apparatus is worn too loosely, and the blood oxygen saturation measurement apparatus needs to be attached tightly to the object.

Optionally, in response to determining that the pressure measurement value is greater than the upper limit of the preset range, e.g., the fourth preset threshold, the second pressure adjustment signal is sent. The second pressure adjustment signal is configured to prompt that the blood oxygen saturation measurement apparatus is worn too tightly, and the blood oxygen saturation measurement apparatus needs to be loose to a certain extent so as to be attached to the object properly.

According to an implementation of the disclosure, the method further includes: in response to determining that the pressure measurement value is not within the preset range, adjusting the pressure between the blood oxygen saturation measurement apparatus and the object until the pressure measurement value is within the preset range.

According to an implementation of the disclosure, the preset range is determined before the measurement, and the step of determining the above preset range includes:

obtaining a first error threshold and a second error threshold of the blood oxygen saturation measurement apparatus, in which the first error threshold is less than the second error threshold. The first error threshold is an upper limit of error for accurate blood oxygen saturation measurement, for example, the first error threshold is used for determining the ideal pressure range, and the second error threshold is an upper limit of error for acceptable blood oxygen saturation measurement, for example, the second error threshold is used for determining the measurable pressure range.

The step of determining the above preset range further includes: obtaining a blood oxygen saturation measurement standard value. The blood oxygen saturation measurement standard value, for example, refers to that, during determination of the above preset range, a user simultaneously uses the blood oxygen saturation measurement apparatus to be calibrated and a high-precision oximeter to measure the blood oxygen saturation value in real time, and the blood oxygen saturation measurement value measured by the high-precision oximeter is determined as the blood oxygen saturation measurement standard value.

Optionally, a pressure value S between the blood oxygen saturation measurement apparatus and the object is adjusted, and a difference between the measured value of the blood oxygen saturation measurement apparatus and the blood oxygen saturation measurement standard value is determined. Further, a change of the difference with the adjustment of the pressure is determined, and the difference is compared with the first error threshold or the second error threshold. For example, if an absolute value E of the difference between the blood oxygen saturation measurement value and the blood oxygen saturation measurement standard value equals to the second error threshold τ2, or approximately the second error threshold τ2, and the pressure value S is inversely correlated with the absolute value E, that is, if the pressure value S increases, the absolute value E decreases, and if the pressure value S decreases, the absolute value E increases. It is determined that the pressure value S is an optional lower limit S1 of the preset range, such as the first preset threshold, or it is determined that the pressure value S is a lower limit of the measurable pressure range.

It can be understood that, in some implementations, the above process may be performed for multiple times to obtain multiple optional lower limits S1 of the preset range, and an average value of the multiple optional lower limits S1 is set as the lower limit S1 of the preset range. The multiple optional lower limits S1 may be determined from measurements on a same user, so that a personalized preset range is determined for the user. In some implementations, the multiple optional lower limits S1 may be determined from measurement on a number of users, wherein the users may be randomly chosen, or the users belong to a same user group.

For instance, the absolute value E of the difference between the blood oxygen saturation measurement value and the measurement standard value is about a second error threshold τ2, for example, the absolute value E is within a range of τ2±τ2×10%. In some implementations, the second error threshold τ2 is 4%, where “%” is a concentration unit of blood oxygen saturation.

For another example, during the adjustment of the pressure value S between the blood oxygen saturation measurement apparatus and the object, the absolute value E of the difference between the blood oxygen saturation measurement value S and the blood oxygen saturation measurement standard value equals to the first error threshold τ1 or approximately the first error threshold τ1. The pressure value S is inversely correlated with the absolute value E, that is, if the pressure value S increases, the absolute value E decreases, and if the pressure value S decreases, the absolute value E increases. In this case, it is determined that the pressure value is an optional lower limit of the ideal pressure range, for example, an optional upper limit S2 of the first preset range, which is the second preset threshold.

In the above example, the absolute value E may be within a range of τ1±τ1×10%. For instance, the first error threshold τ1 is 2%.

It can be understood that, the above process may be performed for multiple times to obtain multiple optional upper limits S2 of the first preset range, and an average value of the multiple optional upper limits S2 of the first preset range is set as the upper limit S2 of the first preset range.

For another example, the pressure value S between the blood oxygen saturation measurement apparatus and the object is adjusted, and the absolute value E of the difference between the blood oxygen saturation measurement value and the blood oxygen saturation measurement standard value equals to the first error threshold or approximately the first error threshold. The pressure value S is positively correlated with the absolute value E, that is, the absolute value E increases in response to the increase of the pressure value S, and the absolute value E decreases in response to the decrease of the pressure value S. In this case, it is determined that the pressure value S is an upper limit of the ideal pressure range or an optional lower limit S3 of the third preset range, which is the third preset threshold.

It can be understood that, the above process can be performed for multiple times to obtain multiple optional lower limits S3 of the third preset range, and an average value of the multiple optional lower limits S3 of the third preset range is set as the lower limit S3 of the third preset range.

For another example, the pressure value S between the blood oxygen saturation measurement apparatus and the object is adjusted until the absolute value of the difference between the blood oxygen saturation measurement value and the measurement standard value equals to the second error threshold or approximately the second error threshold. The pressure value S is positively correlated with the absolute value E, that is, if the pressure value S increases, the absolute value E increases, and if the pressure value S decreases, the absolute value E decreases. In this case, the pressure value S is an optional upper limit of the measurable pressure range or an optional upper limit S4 of the third preset range, that is, the fourth preset threshold.

It can be understood that, the above process can be performed for multiple times to obtain multiple optional upper limits S4 of the third preset range, and the average value of the optional upper limits S4 of the third preset range is set as the upper limit S4 of the third preset range.

In some implementations, a PPG perfusion index can be used to replace the first preset threshold, the second preset threshold, the third preset threshold and the fourth preset threshold as a parameter for starting or evaluating the blood oxygen measurement. Through statistical analysis of a large number of experimental data, the range of the perfusion index that can be used to reliably measure the blood oxygen saturation can be obtained. The above solution does not need to rely on the pressure sensor as the basis for adjustment, which reduces the hardware cost of the blood oxygen saturation measurement apparatus.

In addition, in some implementations, obtaining the blood oxygen saturation measurement standard value includes: obtaining the blood oxygen saturation measurement standard value by at least one high-precision blood oxygen saturation measurement apparatus. Optionally, to obtain the blood oxygen saturation measurement standard value, the blood oxygen saturation measurement value of the object can be obtained separately through multiple high-precision measurement apparatus, and then an average value of the obtained measurement values can be obtained and determined as the measurement standard value. The blood oxygen saturation measurement apparatus is calibrated to satisfy the need for higher accuracy for the individual user. Meanwhile, a large number of users can also be tested, and test values that are commonly used can be selected as the first preset threshold, the second preset threshold, the third preset threshold and/or the fourth preset threshold to meet the needs of ordinary users.

In some implementations, before obtaining the blood oxygen saturation measurement value of the object, signal processing is performed on the blood oxygen saturation measurement signal. The signal processing includes such as, for example, at least one of frequency domain signal processing, time domain signal processing or space domain signal processing. One or more types of signal processing chosen from the group of the frequency domain signal processing, the time domain signal processing and the space domain signal processing are performed on the blood oxygen saturation measurement signal, or other types of signal processing is performed on the blood oxygen saturation measurement signal.

In one example, performing the signal processing on the blood oxygen saturation measurement signal includes: obtaining a first blood oxygen saturation measurement signal with a frequency less than a frequency threshold by performing low-pass filtering on the blood oxygen saturation measurement signal. In another example, performing the signal processing on the blood oxygen saturation measurement signal includes: selecting a second blood oxygen saturation measurement signal with a time domain stability parameter less than a stability threshold from the blood oxygen saturation measurement signal. In another example, performing the signal processing on the blood oxygen saturation measurement signal includes: selecting a third blood oxygen saturation measurement signal with a signal peak amplitude parameter less than an amplitude threshold from the blood oxygen saturation measurement signal. In yet another example, the above three types of signal processing is performed on the blood oxygen saturation measurement signal sequentially, e.g., in the frequency domain, the low-passing filtering is performed on the blood oxygen saturation measurement signal to obtain a first blood oxygen saturation measurement signal, and then in the time domain, signal selection utilizing the time domain stability parameter is performed on the first blood oxygen saturation measurement signal to obtain the second blood oxygen saturation measurement signal, and then in the frequency domain, signal selection utilizing the signal peak amplitude parameter is performed on the second blood oxygen saturation measurement signal to obtain the third blood oxygen saturation measurement signal.

In one implementation, the frequency domain signal processing includes: performing low-pass filtering processing on the blood oxygen saturation measurement signal, in which a cut-off frequency θ_{f} of the low-pass filtering processing is a first preset frequency. The first preset frequency θ_{f} is optically determined based at least part on the pressure measurement value. For example, if the pressure measurement value is within the ideal pressure range, the first preset frequency θ_{f} is set to a value to enable most or all of the blood oxygen saturation measurement signal to be chosen. For another example, if the pressure measurement value is not within the ideal pressure range, e.g., is within the measurable pressure range, the first preset frequency θ_{f} is set to a value to enable a reasonable part of the blood oxygen saturation measurement signal to be chosen, and the other part with a relatively high noise is not used to determine the blood oxygen saturation measurement value.

In another implementation, the time domain signal processing includes: obtaining a time domain stability parameter of the blood oxygen saturation measurement signal, and obtaining the first blood oxygen saturation measurement signal with a time domain stability parameter less than a first stability threshold from the blood oxygen saturation measurement signal. Optionally, the blood oxygen saturation measurement apparatus includes two or more optical signal channels and obtains two optical signals, and the stability of the optical signal can be calculated through a sliding window, and the corresponding threshold θₜ is used to perform signal selection. For example, the two optical signals in the sliding window are R and N respectively, and the stability parameter E is determined based on the following formula:

a=Σ(rᵢ−μ_{R})×(nᵢ−μ_{N})/Σ(nᵢ−μ_{N})²,

E=MSE(a×(N−μ_{N})−(R−μ_{R}))/MSE(R−μ_{R}),

where rᵢ∈R, nᵢ∈N, μ_{N} and μ_{R} are mean values of N and R respectively, and MSE( ) is a mean square error function. If E>θₜ, the blood oxygen saturation measurement signal in the sliding window is considered to be a bad signal and is not used to calculate the blood oxygen saturation measurement value.

In another example, the space domain signal processing includes: obtaining signal peaks of the blood oxygen saturation measurement signal, and obtaining the third blood oxygen saturation measurement signal with a signal peak amplitude parameter less than a first amplitude threshold θₛ from the blood oxygen saturation measurement signal. Firstly, the signal peaks of the blood oxygen saturation measurement signal are extracted, and reference median filtering result of peak amplitudes is obtained by performing median filtering processing on the peak amplitudes, and signal selection is then performed based on the first amplitude threshold θs. For instance, the peak numbers are set as i=1, 2, . . . m, and its corresponding amplitude is pᵢ, and a signal peak amplitude parameter E is determined by the following formula:

E=|pᵢ−median(pⱼ|i−1≤j≤i+1)|,

where median( ) is a median function. If E>θₛ, a signal segment corresponding to pi is considered to be a bad signal, and is not used to calculate the blood oxygen saturation measurement value.

In some implementations, comparing to the case where the pressure measurement value is not in the ideal measurement range, if the pressure measurement value is in the ideal measurement range, the signal processing has a relaxed signal selection rule so that more part of the blood oxygen saturation measurement signal is used to perform the blood oxygen saturation calculation, e.g., by increasing the values of θ_{f}, θₜ and θₛ. In other implementations, if the pressure measurement value is in the ideal measurement range, a part or all of the above signal selection processing is not performed on the blood oxygen saturation measurement signal and all of the blood oxygen saturation measurement signal is used to perform the blood oxygen saturation calculation.

Optionally, after the blood oxygen saturation signal is processed through the above steps, the processed blood oxygen saturation signal is converted into the blood oxygen saturation measurement value. The existing algorithm may be used to convert the processed blood oxygen saturation signal into the blood oxygen saturation measurement value, which will not be repeated in the disclosure.

In an implementation, when the pressure measurement value is in the second preset range, that is, the ideal measurement range, the first type of signal processing is performed on the blood oxygen saturation measurement value, in which the frequency threshold used in the first type of signal processing is the first frequency threshold, the stability threshold used in the first type of signal processing is the first stability threshold, and the amplitude threshold used in the first type of signal processing is the first amplitude threshold.

When the pressure measurement value is in the first preset range or the third preset range that is, a measurable range, the second type of signal processing is performed on the blood oxygen saturation measurement value, in which the frequency threshold used in the second type of signal processing is the second frequency threshold, the stability threshold is the second stability threshold, and the amplitude threshold is the second amplitude threshold.

The first frequency threshold is greater than the second frequency threshold, the first stability threshold is greater than the second stability threshold, and the first amplitude threshold is greater than the second amplitude threshold.

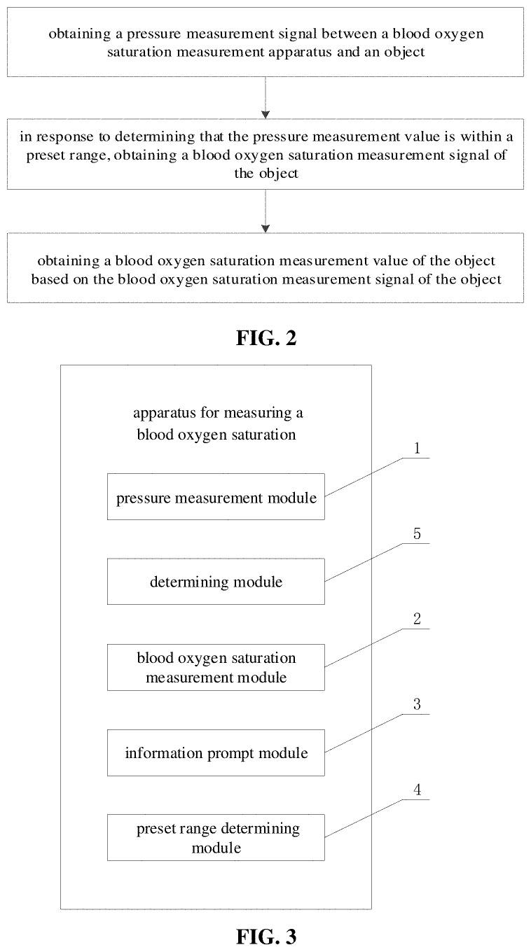

FIG. 3 is a schematic diagram of modules of an apparatus for measuring a blood oxygen saturation according to an implementation of the disclosure.

As illustrated in FIG. 3, the second aspect of implementations of the disclosure provides an apparatus for measuring a blood oxygen saturation. The apparatus includes: a pressure measurement module 1 and a blood oxygen saturation measurement module 2. The pressure measurement module 1 is configured to obtain a pressure measurement signal between a blood oxygen saturation measurement apparatus and an object, so as to obtain a pressure measurement value. The blood oxygen saturation measurement module 2 is configured to obtain a blood oxygen saturation measurement signal of the object in response to determining that the pressure measurement value is within a preset range, and to obtain a blood oxygen saturation measurement value of the object based on the blood oxygen saturation measurement signal of the object.

According to an implementation of the disclosure, the apparatus further includes: an information prompt module 3, configured to, in response to determining that the pressure measurement value is not within the preset range, issue an information prompt signal, in which the information prompt signal is configured to instruct to adjust a pressure between the blood oxygen saturation measurement apparatus and the object.

According to an implementation of the disclosure, the apparatus further includes: a pressure adjustment module, configured to, in response to determining that the pressure measurement value is not within the preset range, adjust a pressure between the blood oxygen saturation measurement apparatus and the object.

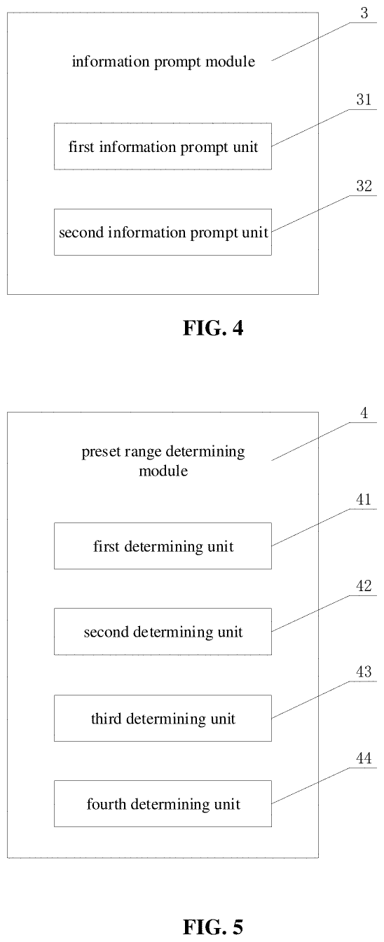

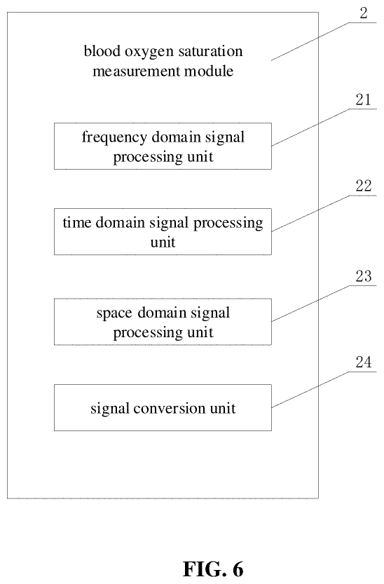

FIG. 4 is a schematic diagram of an information prompt module according to an implementation of the disclosure.

As illustrated in FIG. 4, the information prompt module 3 includes a first information prompt unit 31 and a second information prompt unit 32.

The first information prompt unit 31 is configured to, in response to determining that the pressure measurement value is less than a lower limit of the preset range, issue a first information prompt signal, in which the first information prompt signal is configured to instruct to increase the pressure between the blood oxygen saturation measurement apparatus and the object.

The second information prompt unit 32 is configured to, in response to determining that the pressure measurement value is greater than an upper limit of the preset range, issue a second information prompt signal, in which the second information prompt signal is configured to instruct to reduce the pressure between the blood oxygen saturation measurement apparatus and the object.

According to an implementation of the disclosure, the apparatus further includes: a preset range determining module 4. The preset range determining module 4 is configured to: obtain a first error threshold and a second error threshold of the blood oxygen saturation measurement, in which the first error threshold is less than the second error threshold; obtain a blood oxygen saturation measurement standard value; and determine the preset range based on a difference between the blood oxygen saturation measurement value and the blood oxygen saturation measurement standard value, the first error threshold and the second error threshold.

According to an implementation of the disclosure, the preset range includes: a first preset range, a second preset range and a third preset range. An upper limit of the first preset range is equal to a lower limit of the second preset range, an upper limit of the second preset range is equal to a lower limit of the third preset range.

FIG. 5 is a schematic diagram of a preset range determining module according to an implementation of the disclosure.

As illustrated in FIG. 5, the preset range determining module 4 includes: a first determining unit 41 and a second determining unit 42.

The first determining unit 41 is configured to, in response to adjusting a pressure value between the blood oxygen saturation measurement apparatus and the object, an absolute value of the difference between the blood oxygen saturation measurement value and the blood oxygen saturation measurement standard value being the second error threshold, and the pressure value being inversely correlated with the absolute value, determine the pressure value as a lower limit of the first preset range.

The second determining unit 42 is configured to, in response to adjusting a pressure value between the blood oxygen saturation measurement apparatus and the object, an absolute value of the difference between the blood oxygen saturation measurement value and the blood oxygen saturation measurement standard value being the first error threshold, and the pressure value being inversely correlated with the absolute value, determine the pressure value as the upper limit of the first preset range.

According to an implementation of the disclosure, the preset range determining module 4 includes: a third determining unit 43 and a fourth determining unit 44.

The third determining unit 43 is configured to, in response to adjusting the pressure value between the blood oxygen saturation measurement apparatus and the object, the absolute value of the difference between the blood oxygen saturation measurement value and the blood oxygen saturation measurement standard value being the first error threshold, and the pressure value being positively correlated with the absolute value, determine the pressure value as the lower limit of the third preset range.

The fourth determining unit 44 is configured to, in response to adjusting the pressure value between the blood oxygen saturation measurement apparatus and the object, the absolute value of the difference between the blood oxygen saturation measurement value and the blood oxygen saturation measurement standard value being the second error threshold, and the pressure value being positively correlated with the absolute value, determine the pressure value as an upper limit of the third preset range.

The first error threshold is the upper limit of error for accurate measurement, the second error threshold is the upper limit of error for acceptable measurement, and the blood oxygen saturation measurement standard value is the blood oxygen saturation measurement value in an ideal state.

Optionally, the blood oxygen saturation measurement standard value is the standard value of blood oxygen saturation measured in an ideal state by at least one high-precision blood oxygen saturation measurement apparatus.

According to an implementation of the disclosure, the apparatus further includes: a determining module 5, configured to determine whether the pressure measurement value is within the preset range, and in response to the pressure measurement value being within the preset range, instruct the blood oxygen saturation measurement module 2 to obtain the blood oxygen saturation measurement signal.

According to an implementation of the disclosure, the blood oxygen saturation measurement module 2 is further configured to: perform signal processing on the blood oxygen saturation measurement signal, in which the signal processing includes at least one of frequency domain signal processing, time domain signal processing and space domain signal processing.

According to an implementation of the disclosure, the blood oxygen saturation measurement module 2 includes: a frequency domain signal processing unit 21, a time domain signal processing unit 22, a space domain signal processing unit 23 and a signal conversion unit 24.

The frequency domain signal processing unit 21 is configured to obtain a first blood oxygen saturation measurement signal with a frequency less than a frequency threshold by performing low-pass filtering on the blood oxygen saturation measurement signal.

The time domain signal processing unit 22 is configured to determine a second blood oxygen saturation measurement signal with a time domain stability parameter less than a stability threshold from the first blood oxygen saturation measurement signal.

The space domain signal processing unit 23 is configured to determine a third blood oxygen saturation measurement signal with a signal peak amplitude parameter less than an amplitude threshold from the second blood oxygen saturation measurement signal.

The signal conversion unit 24 is configured to obtain the blood oxygen saturation measurement value based on the third blood oxygen saturation measurement signal.

According to an implementation of the disclosure, the pressure measurement module 1 is configured to, in response to the pressure measurement value being in the second preset range, instruct the blood oxygen saturation measurement module 2 to perform a first type of signal processing on the blood oxygen saturation measurement value. The pressure measurement module 1 is configured to, in response to the pressure measurement value being in the first preset range or the third preset range, instruct the blood oxygen saturation measurement module 2 to perform a second type of signal processing on the blood oxygen saturation measurement value. The blood oxygen saturation measurement module 2 is configured such that a frequency threshold of the first type of signal processing is greater than a frequency threshold of the second type of signal processing, a stability threshold of the first type of signal processing is greater than a stability threshold of the second type of signal processing, and an amplitude threshold of the first type of signal processing is greater than an amplitude threshold of the second type of signal processing.

According to a third aspect of the disclosure, a computer storage medium having computer programs stored thereon is provided. When the computer programs are executed by a processor, the above blood oxygen saturation measurement method is implemented.

According to a fourth aspect of the disclosure, an electronic device is provided. The electronic device includes: a memory, a processor, and computer programs stored on the memory and capable of running on the processor. When the computer programs are executed by a processor, the above blood oxygen saturation measurement method is implemented.

The disclosure provides a blood oxygen saturation measurement method. The method includes: obtaining a pressure measurement signal between a blood oxygen saturation measurement apparatus and an object, so as to obtain a pressure measurement value; in response to determining that the pressure measurement value is within a preset range, obtaining a blood oxygen saturation measurement signal of the object; and obtaining a blood oxygen saturation measurement value of the object based on the blood oxygen saturation measurement signal of the object. The disclosure further provides a blood oxygen saturation measurement apparatus, a storage medium and an electronic device. The above technical solution has the following beneficial effects.

According to the method and apparatus for measuring a blood oxygen saturation of the implementations of the disclosure, the pressure measurement value between the blood oxygen saturation measurement apparatus and the object is obtained, and the pressure value between the blood oxygen saturation measurement apparatus and the object is adjusted when the pressure measurement value is not in the preset range, and the blood oxygen saturation information is acquired when the pressure measurement value is within the preset range, which avoids the acquisition of erroneous blood oxygen saturation information, and improves the accuracy of measurement by the blood oxygen saturation measurement apparatus.

It should be understood that the above specific implementations of the disclosure are only used to illustrate or explain the principle of the disclosure, but not to limit the disclosure. Therefore, any modifications, equivalent replacements and improvements made without departing from the spirit and scope of the disclosure should be included in the protection scope of the disclosure. Furthermore, the appended claims of the disclosure are intended to cover all changes and modifications that fall within the scope and boundaries of the appended claims, or the equivalents of such scope and boundaries.

1. A method for measuring a blood oxygen saturation by a blood oxygen saturation measurement apparatus, comprising:
obtaining a pressure measurement value based on a pressure measurement signal between the blood oxygen saturation measurement apparatus and an object;
in response to determining that the pressure measurement value is within a preset range, obtaining a blood oxygen saturation measurement signal of the object; and
obtaining a blood oxygen saturation measurement value of the object based on the blood oxygen saturation measurement signal of the object.

2. The method of claim 1, further comprising:
in response to determining that the pressure measurement value is not within the preset range, adjusting a pressure between the blood oxygen saturation measurement apparatus and the object until an obtained pressure measurement value is within the preset range.

3. The method of claim 1, further comprising:
obtaining a first error threshold and a second error threshold of the blood oxygen saturation measurement apparatus, wherein the first error threshold is less than the second error threshold;
obtaining a blood oxygen saturation measurement standard value; and
determining the preset range based at least in part on the blood oxygen saturation measurement standard value, the first error threshold and the second error threshold.

4. The method of claim 3, wherein determining the preset range based at least in part on the blood oxygen saturation measurement standard value, the first error threshold and the second error threshold comprises:
determining the preset range based on a change of a difference between a measured blood oxygen saturation measurement value and the blood oxygen saturation measurement standard value with a change of a pressure between the blood oxygen saturation measurement and the object, as well as based on a comparison result of the difference and at least one of the first error threshold and the second error threshold.

5. The method of claim 1, wherein obtaining the blood oxygen saturation measurement value of the object based on the blood oxygen saturation measurement signal of the object comprises:
performing at least one type of signal processing on the blood oxygen saturation measurement signal based on the pressure measurement value.

6. The method of claim 5, wherein the blood oxygen saturation measurement signal of the object comprises a first blood oxygen saturation measurement signal and a second blood oxygen saturation measurement signal; and
wherein performing at least one type of signal processing on the blood oxygen saturation measurement signal comprises:
determining a time domain stability parameter indicating a correlation between the first blood oxygen saturation measurement signal and the second blood oxygen saturation measurement signal; and
performing signal selection processing on the blood oxygen saturation measurement signal based on the time domain stability parameter, wherein a parameter of the signal selection processing is determined based at least part on the pressure measurement value.

7. The method of claim 5, wherein performing at least one type of signal processing on the blood oxygen saturation measurement signal comprises:
performing low-pass filtering processing on the blood oxygen saturation measurement signal, wherein a cut-off threshold of the low-pass filtering processing is determined based at least part on the pressure measurement value.

8. The method of claim 5, wherein performing at least one type of signal processing on the blood oxygen saturation measurement signal comprises:
performing median filtering processing on peak amplitudes of the blood oxygen saturation measurement signal to obtain a filtering processing result; and
performing signal selection processing on the blood oxygen saturation measurement signal based on the filtering processing result, wherein a parameter of the signal selection processing is determined based at least part on the pressure measurement value.

9. The method of claim 5, wherein performing at least one type of signal processing on the blood oxygen saturation measurement signal comprises at least one of:
in response to the pressure measurement value being in an ideal pressure range, performing first signal processing on the blood oxygen saturation measurement value; or
in response to the pressure measurement value being not in the ideal pressure range, performing second signal processing on the blood oxygen saturation measurement value;
wherein the preset range comprises the ideal pressure range, and the first signal processing and the second signal processing have different processing parameters.

10. The method of claim 5, wherein performing at least one type of signal processing on the blood oxygen saturation measurement signal comprises at least one of:
in response to the pressure measurement value being in an ideal pressure range, utilizing all of the blood oxygen saturation measurement signal to obtain the blood oxygen saturation measurement value; or
in response to the pressure measurement value being not in the ideal pressure range, performing signal selection processing on the blood oxygen saturation measurement signal;
wherein the preset range comprises the ideal pressure range.

11. An apparatus for measuring a blood oxygen saturation, comprising:
a processor; and
a memory for storing instructions executable by the processor, wherein the processor is configured to execute the instructions stored in the memory to:
obtain a pressure measurement signal between a blood oxygen saturation measurement apparatus and an object, so as to obtain a pressure measurement value;
in response to determining that the pressure measurement value is within a preset range, obtain a blood oxygen saturation measurement signal of the object; and
obtain a blood oxygen saturation measurement value of the object based on the blood oxygen saturation measurement signal of the object.

12. The apparatus of claim 11, wherein the processor is further configured to execute the instructions stored in the memory to:
in response to determining that the pressure measurement value is not within the preset range, adjust a pressure between the blood oxygen saturation measurement apparatus and the object.

13. The apparatus of claim 12, wherein the instructions to, in response to determining that the pressure measurement value is not within the preset range, adjust the pressure between the blood oxygen saturation measurement apparatus and the object, comprise instructions to:
in response to determining that the pressure measurement value is less than a lower limit of the preset range, increase the pressure between the blood oxygen saturation measurement apparatus and the object; or
in response to determining that the pressure measurement value is greater than an upper limit of the preset range, reduce the pressure between the blood oxygen saturation measurement apparatus and the object.

14. The apparatus of claim 11, wherein the processor is further configured to execute the instructions stored in the memory to:
obtain a first error threshold and a second error threshold of the blood oxygen saturation measurement, wherein the first error threshold is less than the second error threshold;
obtain a blood oxygen saturation measurement standard value; and
determine the preset range based on a difference between the blood oxygen saturation measurement value and the measurement standard value, the first error threshold and the second error threshold.

15. The apparatus of claim 14, wherein the preset range comprises: a first preset range, a second preset range and a third preset range, wherein an upper limit of the first preset range is equal to a lower limit of the second preset range, and an upper limit of the second preset range is equal to a lower limit of the third preset range.

16. The apparatus of claim 15, wherein the instructions to determine the first preset range comprise instructions to:
in response to an absolute value of the difference between the blood oxygen saturation measurement value and the measurement standard value being the second error threshold, and a pressure value between the blood oxygen saturation measurement apparatus and the object being inversely correlated with the absolute value, determine the pressure value as a lower limit of the first preset range; or
in response to an absolute value of the difference between the blood oxygen saturation measurement value and the measurement standard value being the first error threshold, and the pressure value being inversely correlated with the absolute value, determine the pressure value as the upper limit of the first preset range.

17. The apparatus of claim 15, wherein the instructions to determine the third preset range comprise instructions to:
in response to the absolute value of the difference between the blood oxygen saturation measurement value and the measurement standard value being the first error threshold, and a pressure value between the blood oxygen saturation measurement apparatus and the object being positively correlated with the absolute value, determine the pressure value as the lower limit of the third preset range; or
in response to the absolute value of the difference between the blood oxygen saturation measurement value and the measurement standard value being the second error threshold, and the pressure value being positively correlated with the absolute value, determine the pressure value as an upper limit of the third preset range.

18. The apparatus of claim 15, wherein the processor is further configured to execute the instructions stored in the memory to:
before obtaining the blood oxygen saturation measurement value of the object, perform signal processing on the blood oxygen saturation measurement signal, wherein the signal processing comprises at least one of frequency domain signal processing, time domain signal processing and space domain signal processing.

19-20. (canceled)

21. The apparatus of claim 18, wherein the instructions to perform the signal processing on the blood oxygen saturation measurement signal comprise instructions to:
obtain a first blood oxygen saturation measurement signal with a frequency less than a frequency threshold by performing low-pass filtering on the blood oxygen saturation measurement signal;
determine a second blood oxygen saturation measurement signal with a time domain stability parameter less than a stability threshold from the first blood oxygen saturation measurement signal; and
determine a third blood oxygen saturation measurement signal with a signal peak amplitude parameter less than an amplitude threshold from the second blood oxygen saturation measurement signal.

22. The apparatus of claim 11, further comprising:
a pressure sensor, configured to obtain the pressure measurement signal between the blood oxygen saturation measurement apparatus and the object; and
a blood oxygen saturation sensor, configured to obtain the blood oxygen saturation measurement signal of the object.

23. A non-transitory computer-readable storage medium, having stored therein instructions that, when executed by a processor of an electronic device, causes the electronic device to perform the method according to claim 1.
